(19) United States
(12) Patent Application Publication
(10) Pub. No.: US 2006/0094664 A1
(43) Pub. Date: May 4, 2006

(54) Deoxycholic acid derivatives for the treatment of acute dysfunctions of portal and hepatic venous circulation

(76) Inventors: Piero Del Soldato, Monza (Milano) (IT); Giancarlo Acuto, Castelmarte (CO) (IT)

Correspondence Address: ARENT FOX PLLC, 1050 CONNECTICUT AVENUE, N.W., SUITE 400, WASHINGTON, DC, 20036, US

(21) Appl. No.: 10/512,856

(22) PCT Filed: May 9, 2003
(86) PCT No.: PCT/EP03/04861

§ 371 Date: May 19, 2005

(30) Foreign Application Priority Data
May 14, 2002 (IT) MI2002A001025

(51) Int. Cl. C07J 17/00; A61K 38/05; A61K 31/58

(52) U.S. Cl. 514/18; 514/172; 514/26; 514/176; 530/331; 536/6.1; 540/107

(57) ABSTRACT

Use of compounds and their salts of formula (I), as reported in the description, in the preparation of drugs for the treatment of acute dysfunctions of portal and hepatic venous circulation.

The present invention relates to the use of drugs for the acute treatment of hepatic and portal venous circulation disorders or hemodynamic decompensation.

More particularly, the present invention relates to the use of drugs for the acute treatment of hepato-portal tract disorders and not for the chronic treatment of hepatic diseases, such as for example cirrhosis.

The hepatic and portal venous circulation disorders are characterized by an improved intrahepatic flow resistance or by an increase of portal vein flow, due to a vessel occlusion, or congestion, generally caused by a liver disorder. The acute treatment is only directed to reduce the increase of the portal pressure, whereas the chronic treatment, that starts in the early phase of the disease, has merely the aim to limit the progress of said disease. The present invention relates to a treatment able to decrease the portal pressure in acute phase. In fact, it is known that should the portal blood flow not be brought back to physiological values, it may have serious clinic consequences for a patient, such as:

development of portosystemic collateral circulation (gastroesophageal varices)

direct shunting of portal blood into vena cava (hepatic encephalopathy)

abdominal viscera congestion (malabsorption) and splenomegaly (hypersplenism with platletspenia)

ascites.

Also in case of chronic liver diseases, such as cirrhosis, hepatitis, cancer, these worsen the portal pressure. In industrialized nations, cirrhosis is by far the most common cause of portal hypertension, although schistosomiasis predominates in some tropical and subtropical climates.

Further factors that may contribute to the appearance of said disorders can be alcohol-related liver damage, congenital hepatic fibrosis, drug poisoning, autoimmune diseases.

Acute bleeding from esophageal varices (usually from distal oesophagus, less often from the gastric fundus and only rarely from other sites) is the most common clinical picture of these hepatic or portal venous circulation disorders. Generally, patients present with sudden painless upper gastrointestinal hemorrhage, often massive. Acute bleeding is a very serious phenomenon that must be treated for avoiding consequences also fatal for the patient.

The pharmacological therapy for the acute treatment of variceal bleeding consists in using drugs able to reduce the portal pressure. Vasopressin, somatostatin and its analogues may be mentioned. However, vasopressin exhibits side effects such as mesenteric and myocardial ischemia. Generally, effectiveness of these drugs in treatment of acute bleeding has not been established.

For this reason, the pharmacological therapy commonly employed for said treatment in acute phase makes use of β-blockers, such as for example propranolol, nadolol, timolol, etc. These drugs can be administered alone or in association with isosorbide mononitrate.

The β-blockers are active in reducing portal flow resistance but exhibit the following collaterals:

they possess side effects on cardiovascular and respiratory system. For this reason, they can not be administered to patients having cardiovascular problems, asthma, COPD (chronic obstructive pulmonary disease) etc.,

in a few subjects intolerance of these drugs occurred, thus developing dyspnoea and bronchospnea, dyspnoea and cardiopathy, asthenia, gastric intolerance and hepatic encefalopathy.

For the acute treatment of hepatic and portal venous circulation disorders also vasodilators have been used, such as for example isosorbide mononitrate. However, their systemic vasodilatatory action may be not well tolerated by patients suffering from portal hypertension, in that they can give rise to a reduction of systemic pressure.

Owing to the side effects exhibited by the above mentioned drugs in the acute varices treatment, surgical techniques such as endoscopic treatment with prophylactic sclerosis of esophageal varices, transjugular intrahepatic portal-systemic shunting or surgical shunting have been employed.

It was thus an object of the present invention to provide drugs effective in the acute treatment of hepatic and portal venous circulation disorders having improved activity and tolerability. The treatments used in chronic phase of liver diseases did not give any suggestion about the treatment of the acute phase, in that the drugs employed in chronic phase act only in the treatment of hepatic diseases. As drugs largely employed for the chronic phase treatment ursodeoxycholic acid (UDCA) and interferon may be mentioned.

Accordingly, the present invention relates to the use for the acute treatment of hepatic or portal venous circulation disorders of compounds having the following formula (I) 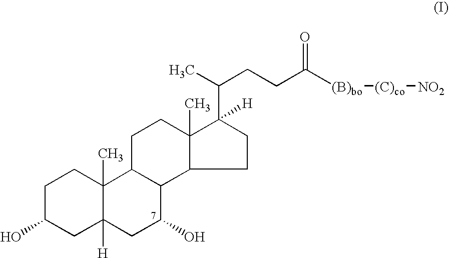
wherein:

the bond between the hydroxylic group and the carbon atom in 7 position is α- or β-standing, in which when said bond is β-standing, the steroidal structure of figure (I) corresponds to the ursodeoxycholic acid residue, whereas when the above bond is α-standing, the steroidal structure corresponds to the chenodeoxycholic acid residue;
b₀=0, 1;
c₀=0, 1, with the proviso that they can not be simultaneously 0;
B=T_{B}-X₂-T_{BI}, wherein T_{B} and T_{BI} are the same or different, and T_{B}=X, wherein X is —O—, —S—, —N(R_{1c}), R_{1c} being H, C₁-C₅ straight or branched alkyl, and T_{BI}=(CO)ₜₓ or (X)ₜₓₓ, wherein tₓ and txx are 0 or 1, with the proviso that tx=1 when txx=0 and tx=0 when txx=1, X being as defined above;
X₂ is a bivalent radical such that the T_{B}-X₂-T_{BI} moiety for B (in which the free valence of T_{B} is saturated with Z, Z being H, C₁-C₁₀ straight or branched alkyl, and the free valence of T_{BI} is saturated with OZ, Z or with —N(Z¹) (Z²), wherein Z¹ and Z² are the same or different and have the meaning mentioned above for Z) when T_{BI}=CO or X, according to the tx and txx values, X being as defined above, is selected from:
   amino acids,
   hydroxy acids,
   mono- or polyalcohols;
C=-T_{c}-Y—, wherein T_{c}=(CO) or X as defined above;
   when b₀=c₀=1: T_{c}=(CO) when tₓ=0, T_{c}=X
   when tₓₓ=0, X being as defined above;
   when b₀=0: T_{c}=X, X being as defined above;
   when c₀=0: tₓ=0, t_{BI}=X =-0-;
Y is selected from:
Yₚ; 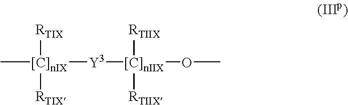
   wherein:
nIX is an integer of from 0 to 10, preferably of from 1 to 3;
nIIx is an integer of from 1 to 10, preferably of from 1 to 3;
R_{TIX}, R_{TIX′}, R_{TIIX}, R_{TIIX′} are the same or different and are H or C₁-C₄ straight or branched alkyl, preferably R_{TIX}, R_{TIX′}, R_{TIIX}, R_{TIIX′} are H;
Y³ is a 5 or 6 member heterocyclic ring comprising one or two heteroatoms selected from nitrogen, oxygen or sulfur, said ring being saturated, unsaturated or aromatic; Y₀, selected from:
   an alkylenoxy group —R′O, wherein R′ is C₁-C₂₀ straight or branched alkyl, preferably with 2-6 carbon atoms, or cycloalkylene with 5-7 carbon atoms, one or more carbon atoms in cycloalkylene ring being eventually replaced by heteroatoms, and the ring having optionally type R′ side chains, in which R′ is as defined above; or one of the following groups: 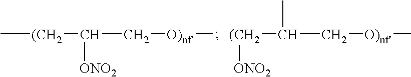
   wherein nf′ is an integer of from 1 to 6, preferably of from 1 to 4 carbon atoms, 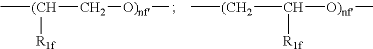
   wherein R_{1f}=H, CH₃ and nf′ is as defined above;
   Y_{Ar}, that is selected from: 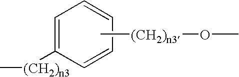
   wherein n3 is an integer of from 0 to 3 and n3′ an integer of from 1 to 3; 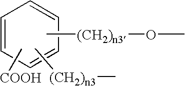
   wherein n3 and n3′ are as defined above.

Preferably the B precursor is selected from the following:
amino acids, preferably selected from L-carnosine (formula CI), anserine (CII), selenocysteine (CIII), selenomethionine (CIV), penicillamine (CV), N-acetylpenicillamine (CVI), cysteine (CVII), N-acetylcysteine (CVIII), glutathione (CIX) or esters thereof, preferably ethyl or isopropyl ester, aspartic acid (PI), hystidine (PII), 5-hydroxytryptophan (PIII):

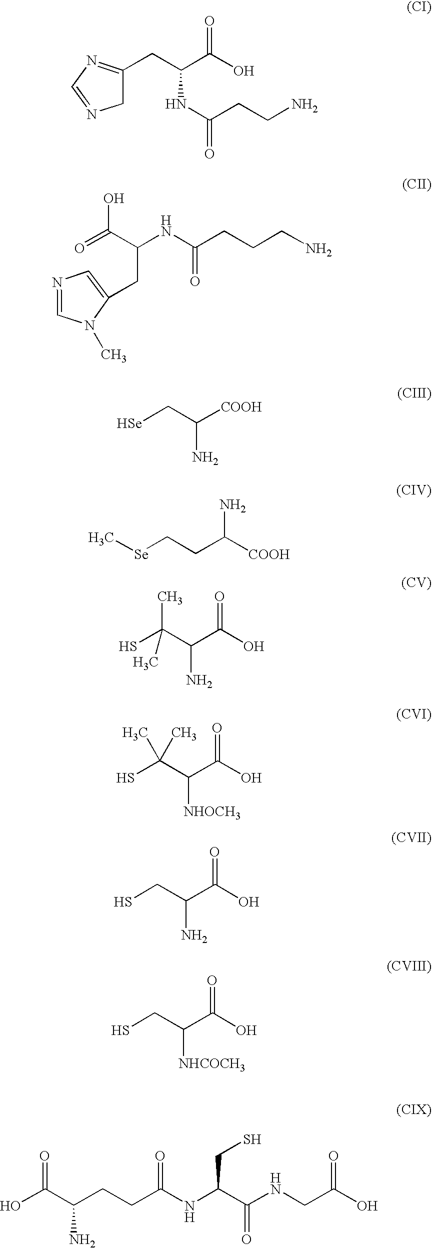

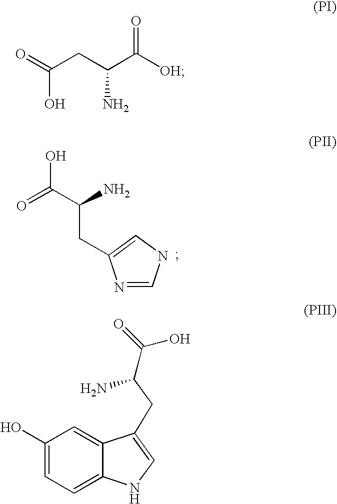

hydroxy acids, preferably selected from the following: gallic acid (DI), ferulic acid (DII), gentisic acid (DIII), citric acid (DIV), caffeic acid (DV), dihydroxycaffeic acid (DVI), p-coumaric acid (DVII), vanillic acid (DVIII), dihydroxymaleic acid (NIII):

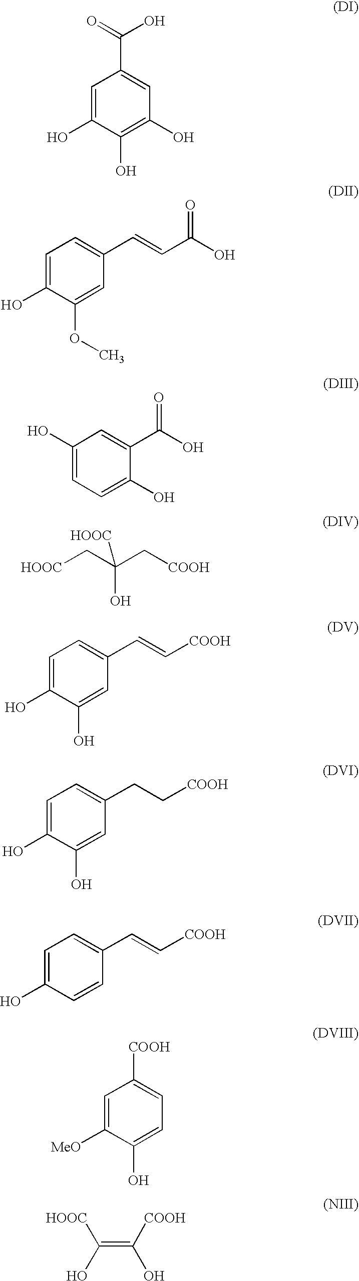

mono or polyalcohols, preferably selected from the following: nordihydroguaiaretic acid (EI), quercetin (EII), catechin (EIII), kaempferol (EIV), sulfuretin (EV), hydroquinone (EVIII), gossypol (EIX), reductic acid (EX), methoxyhydroquinone (EXI), hydroxyhydroquinone (EXII), propyl gallate (EXIII), 3,5-di-ter.butyl-4-hydroxybenzyl-thioglycolate (EXXIV), saccharose (EC), ascorbic (ECI) and isoascorbic (ECII) acid, p-coumaric alcohol (ECIII), 4-hydroxy-phenylethyl alcohol (ECIV), conyferil alcohol (ECV), 2-thiouracil (QI), 2-mercaptoethanol (QII):

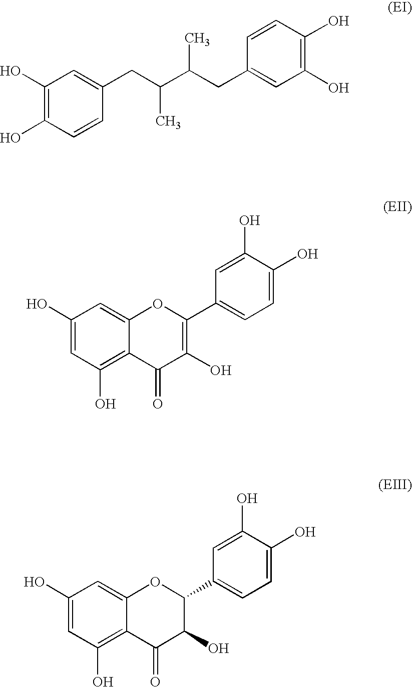

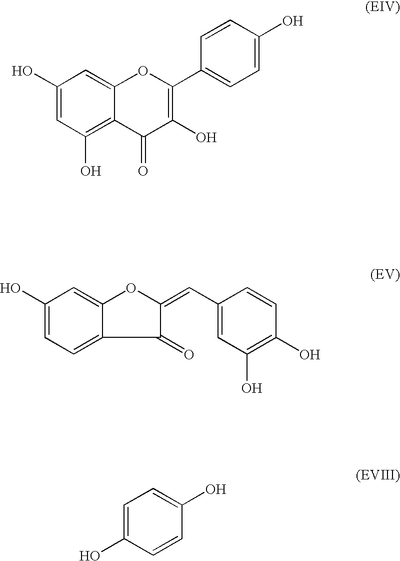

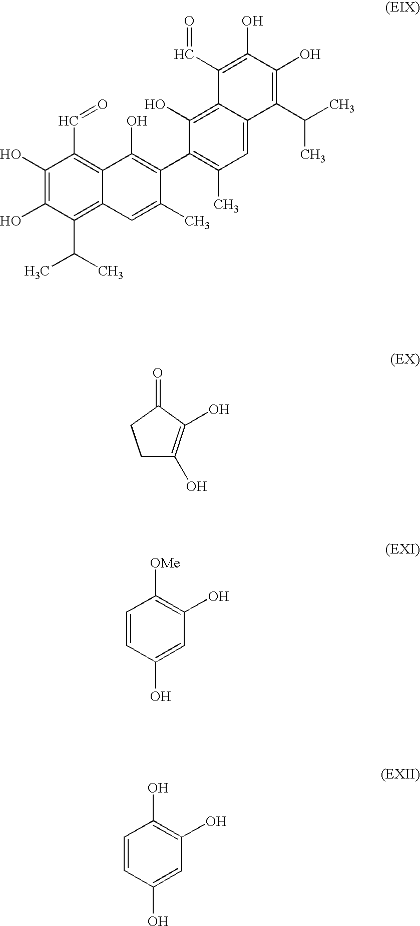

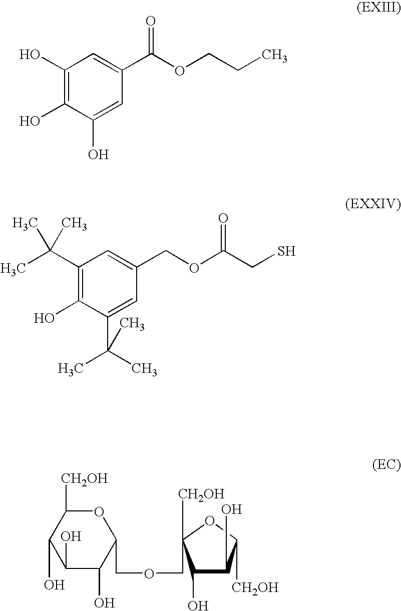

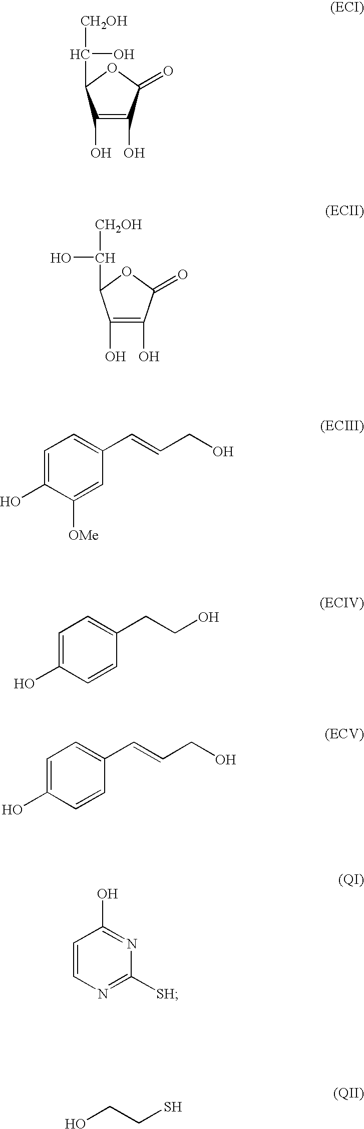

The compounds having the formulae reported above can be obtained according to methods well-known from literature, for example described in “The Merck Index”, 12^{th} Ed. (1996). When available, the corresponding optical or geometrical isomers may be employed.

When b₀=c₀=1 and when b₀=0 and c₀=1, the bonds between the drug radical and X₂ as well as between X₂ and Y can be independently of ester, thioester or amid type.

Preferably Y³ of bivalent radical C is selected from the following bivalent radicals:

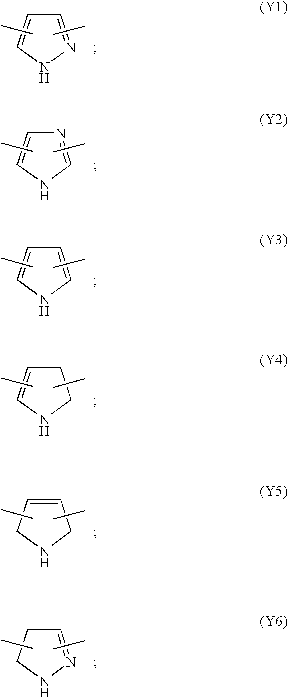

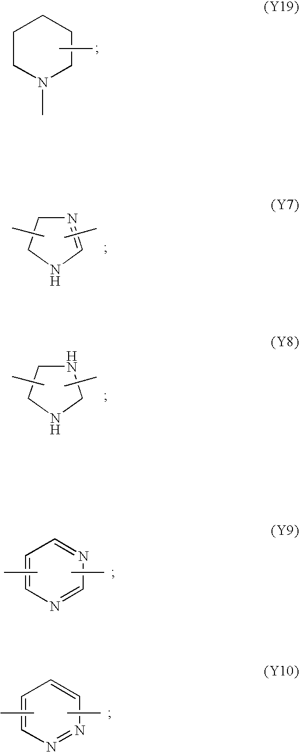

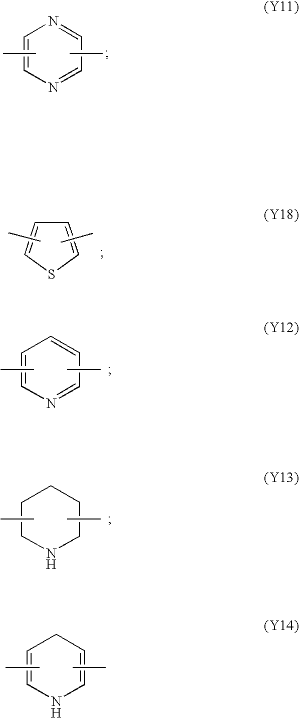

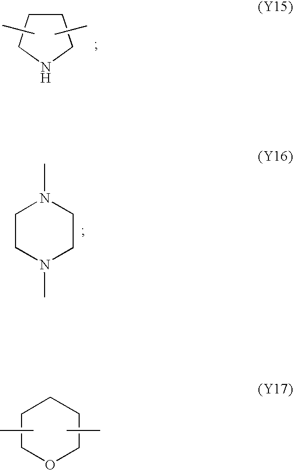

The preferred radicals for Y³ are the following: (Y12), with both the free valences in ortho position as to the nitrogen atom; (Y16) with both the free valences attached to the nitrogen atoms; (Y1), 3,5-disostituted pyrazole; (Y19), wherein the free valence is para-standing on the ring as to the nitrogen atom.

The Y precursors having the formula (III^{p}), in which the free valence on oxygen atom is saturated with H and the free valence on end carbon atom is saturated with a carboxylic or oxydrilic group, are available on the market or they can be prepared according to methods well-known in the art.

The compounds according to the present invention, when at least a functional group that may be salified with acid is present, for example an amine group, can be transformed in the corresponding salts. For example, a process for obtaining salts is the following: when into the molecule a basic nitrogen atom is present, the reaction with an equimolar amount of the corresponding organic or inorganic acid is carried out in an organic solvent, such as acetonitrile, tetrahydrofuran. Examples of organic acids are oxalic, tartaric, maleic, succinic, citric and trifluoroacetic acids.

Examples of inorganic acid are nitric, hydrochloric, sulphuric and phosphoric acids.

Compounds that are employed for the therapeutic uses according to the present invention may be obtained as described for example in WO 00/61604.

When the precursor compounds employed in the present invention have one or more chiral centre, they may be in racemic form or as diastereomers mixture, as single enantiomers or diastereomers. Should geometric asymmetrie be present, the compounds can be used into the cis or trans form.

The compounds object of the present invention are formulated into the corresponding pharmaceutical compositions, also in sustained release form, for parenteral or oral use, for example sublingual, inhalation, transdermic, as suppositories or enema, according to techniques well-known in the art: see for example “Remington's Pharmaceutical Sciences” 15^{th} Ed.

The active ingredient molar amount in said formulations is generally equal or lower than the amount of the corresponding drug precursor.

The daily dose that can be administered is equal to or eventually lower than the dose of the precursor drug. The precursor daily dose can be found for example in “Physician's Desk Reference”.

When B is present in formula (I), that is b0=1, the compounds preferred for the use according to the present invention are those in which B arises from the precursor ferulic acid, in particular the more preferred compound is (3α,5β,7β)-3,7-dihydroxycholan-24-oic acid 2-methoxy-4[3-(4-(nitrooxy)butoxy]-3-oxo-1-propenyl]phenyl ester having the following formula:

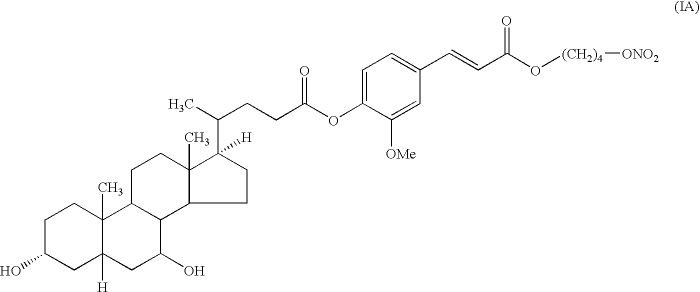

When b0=0 in formula (I), the preferred group Y is of Y₀ type, in particular the alkylene group R′O, R′ being C₃-C₆ alkyl. A particular preferred compound is (3α,5β,7β)-3,7-dihydroxycholan-24-oic acid 4-(nitrooxy)butyl ester of the following formula:

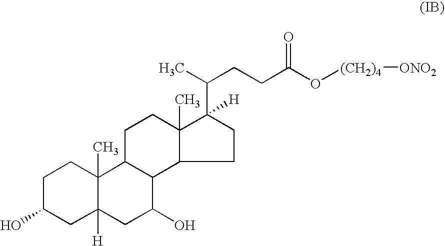

The drugs of the present invention, employed for the acute treatment of hepatic and portal hemodynamic decompensation, possess surprisingly and unexpectedly optimal results in reducing portal pressure. In fact, the precursors of the invention compounds, such as for example ursodeoxycholic acid, are effective in the chronic treatment of hepatic disorders but not for reducing portal pressure after an acute treatment. In the literature it has been never described the use of invention compounds for treating the acute phase of hepatic disorders when a high portal pressure is occurring. In fact, also in case of high portal pressure conditions, for example 500% as to the basal value, induced by a norepinephrine treatment, the invention compounds are able to reduce the portal pressure without influencing the systemic hemodynamic parameters. Unlike the treatment in chronic phase, in the acute treatment the administration of the compounds of the invention is carried out for very short cycles, generally a few days, at most a week, whereas in chronic treatment the administration occurs for long periods of time, at least for eight weeks, sometime months, in that a cirrhosis has been developed in liver. Therefore, it was not foreseeable that treatments having so a short term could be able to show a so high activity in reducing the portal pressure in acute phase treatment.

The following examples are to illustrate but not to restrict the invention.

### EXAMPLES

### Example 1

### Effect of Ursodeoxycholic Acid and of (3α,5β,7β)-3,7-dihydroxycholan-24-oic Acid 2-methoxy-4[3-[4-(nitrooxy)butoxy]-3-oxo-1-propenyl]phenyl Ester in an Experimental Model of Hepatic and Portal Venous Circulation Disorder Induced by Ligature of Biliar Duct and Subsequent Treatment with Norepinephrine.

The ursodeoxycholic acid nitrooxyderivative here employed (NO-urso) has been prepared as described in Example 1 of patent application PCT WO 00/61,604.

Twenty one Wistar rats were divided in three groups of 7 animals each and then subjected to ligature of bile duct. Within three weeks following the ligature, the animals were left on rest. As a consequence of the bile duct ligature, the animals developed dysfunctions of the hepatic and portal venous circulation. At the end of the third week, each group underwent the following weekly treatment protocol:
Group 1: control group treated with carrier (1% w/v water suspension of carboxymethylcellulose);
Group 2: treated with NO-urso at a dose of 28 mg/kg (0.04 mmol/kg), twice a day;
Group 3: treated with ursodeoxycholic acid (urso in table) at a dose of 15 mg/kg (0,04 mmol/kg), twice a day.

The rested compounds have been administered by intragastric cannula in 1% water suspension of carboxymethylcellulose.

At the end of the treatment (fourth week) the animals were anaesthetized with urethane and then systemic pressure (MAP) and portal pressure (PP) were monitored by catheterisation both of the carotid and portal vein. The animal liver was then continuously perfused with Krebs solution (40 ml/min) balanced with O₂/CO₂ (95%-5%) using a peristaltic pump (Gilson) In this way the pharmacological effect of both tested compounds has been evaluated.

The rat liver was then firstly perfused with norepinephrine solution (1 μM) in order to induce an intrahepatic circulation constriction. Afterwards, in groups 2 and 3 a single infusion with a 1 mM solution of each of the tested compounds was carried out and portal pressure variations have been monitored.

The results obtained in the experiments show that the treatments with No-urso or with ursodeoxycholic acid do not influence the systemic hemodynamic parameters, i.e. systemic pressure, cardiac frequency and also hepatic fibrosis evaluated by means of an immunocytochemical method.

The data presented in Table 1 show that NO-urso reduces intrahepatic resistance induced both by bile duct constriction and norepinephrine (NE) administration. In fact, administration of NO-urso unlike that of ursodeoxycholic acid, is able to reduce intrahepatic resistance in animals having high intrahepatic resistance and/or marked hepatic alteration.

After the week treatment, at the perfusion rate of 40 ml/min NO-urso induced a portal pressure reduction of 3 mmHg. The rats treated with Urso showed instead an increase of 2 mmHg with respect to controls. The portal pressure reduction obtained by NO-urso perfusion resulted statistically significant (p<0.01).

The increase of intrahepatic resistance induced by norepinephrine was significantly reduced by NO-urso, but not by ursodeoxycholic acid. In fact, the 500% increase of the intrahepatic vasal resistance, determined by NE infusion at a dose of 1 pM, is significantly reduced by the treatment with NO-urso (p<0.001) but not by ursodeoxycholic acid. This result, together with the observation that the acute treatment with NO-urso does not modify hepatic fibrosis, confirmed that NO-urso acts in this experimental model on dynamic component (vessel pressure) of the intrahepatic resistance increase. The comparison between the obtained data evidences the NO-urso effectiveness in the acute treatment of hemodynamic decompensation of the hepato-portal tract.

### Example 2

### Preparation of (3α,5β,7β)-3,7-dihydroxycholan-24-oic Acid 4-(nitrooxy)butyl Ester (IB)

a) Synthesis of (3α,5β,7β)-3,7-dihydroxycholan-24-oic acid 4-bromobutyl ester

Ursodeoxycholic acid (0.5 g) was reacted with sodium ethylate (0.09 g) in DMF to give the corresponding sodium salt. This solution was dropped into a solution of 1,4-dibromobutane (0.263 g) in DMF. The mixture thus obtained was allowed to stand overnight under stirring at room temperature. The mixture was then extracted with ethyl acetate/water 2:1, the collected organic phases were dried, the solvent was evaporated off at reduced pressure and the residue was purified by silica gel chromatography with n-hexane/ethyl acetate 1:9 as eluent to give 0.1 g of (3α,5β,7β)-3,7-dihydroxycholan-24-oic acid 4-bromobutyl ester.

¹H-NMR (CDCl₃, ppm): 4.12 (2H, t); 3.62 (2H, m); 3.45 (2H, t); 2.3 (2H, m); 1.98-0.96 (36H, m); 0.69 3H, s).
b) Synthesis of (3α,5β,7β)-3,7-dihydroxycholan-24-oic acid 4-(nitrooxy)butyl ester.

To a solution of (3α,5β,7β)-3,7-dihydroxycholan-24-oic acid 4-bromobutyl ester (0.1 g) in acetonitrile (20 ml) silver nitrate was added under stirring (0.066 g) and the mixture was heated for 6 hours under stirring at 80° C. At the end of the reaction, the precipitate thus obtained was filtered off and the solvent removed. The crude product was purified by silica gel chromatography with methylene chloride/ethyl acetate 3/7 as eluent to give 50 mg of the desired compound.

¹NMR (CDCl₃, ppm): 4.3 (2H, m); 4.12 (2H, t); 3.45 (2H, t); 2.3 (2H, m); 1.98-0.96 (36H, m); 0.69 (3H, s).

TABLE 1

| | Portal pressure | | Systemic hemodynamic | |
|---|---|---|---|---|
| | After a | | parameters | |
| | week | % increase | Systemic | |
| | treatment | after NE | pressure | Cardiac |
| | (mmHg) | infusion | (MAP) | frequency |
| Controls | 17 | 500 | 92 ± 3.3 | 358 ± 38 |
| NO-urso | 14* | 180 | 99.6 ± 8.4 | 400 ± 27 |
| Urso | 19 | 400 | 95 ± 12 | 372 ± 41 |

P < 0.01 vs controls

1. Use for the acute treatment of hepatic and portal venous circulation disorders of compounds or salts thereof having the following formula (I) 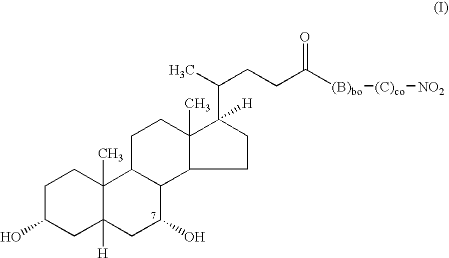
wherein:
the bond between the hydroxylic group and the carbon atom in 7 position is α- or β-standing, in which when said bond is β-standing, the steroidal structure of figure (I) corresponds to the ursodeoxycholic acid residue, whereas when the above bond is α-standing, the steroidal structure corresponds to the chenodeoxycholic acid residue;
b₀=0, 1;
c₀=0, 1, with the proviso that they can not be simultaneously 0;
B=T_{B}-X₂-T_{BI}, wherein T_{B} and T_{BI} are the same or different, and T_{B}=X, wherein X is —O—, —S—, —N(R_{1c}), R_{1c} being H, C₁-C₅straight or branched alkyl, and T_{BI}=(CO)ₜₓ or (X)ₜₓₓ, wherein tₓ and txx are 0 or 1, with the proviso that tx=1 when txx=0 and tx=0 when txx=1, X being as defined above;
X₂ is a bivalent radical such that the T_{B}-X₂-T_{BI} moiety for B (in which the free valence of T_{B} is saturated with Z, Z being H, C₁-C₁₀ straight or branched alkyl, and the free valence of TBI is saturated with OZ, Z or with —N(Z¹)(Z²), wherein Z¹ and Z² are the same or different and have the meaning mentioned above for Z) when T_{BI}=CO or X, according to the tx and txx values, X being as defined above, is selected from:
amino acids,
hydroxy acids,
mono- or polyalcohols;
C=-T_{c}-Y—, wherein T_{c}=(CO) or X as defined above;
when b₀=c₀=1: T_{c}=(CO) when tₓ=0, T_{c}=X
when tₓₓ=0, X being as defined above;
when b₀=0: T_{c}=X, X being as defined above;
when c₀=0: tₓ=0, t_{BI}=X=—O—;
Y is selected from:
Yₚ; 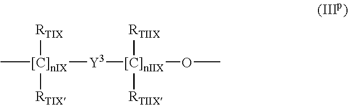
wherein:
nIX is an integer of from 0 to 10, preferably of from 1 to 3;
nIIX is an integer of from 1 to 10, preferably of from 1 to 3;
R_{TIX}, R_{TIX′}, R_{TIIX}, R_{TIIX′} are the same or different and are H or C₁-C₄ straight or branched alkyl, preferably R_{TIX}, R_{TIX′}, R_{TIIX}, R_{TIIX′} are H;
Y³ is a 5 or 6 member heterocyclic ring comprising one or two heteroatoms selected from nitrogen, oxygen or sulfur, said ring being saturated, unsaturated or aromatic;
Y₀, selected from:
an alkylenoxy group —R′O, wherein R′ is C₁-C₂₀ straight or branched alkyl, preferably with 2-6 carbon atoms, or cycloalkylene with 5-7 carbon atoms, one or more carbon atoms in cycloalkylene ring being eventually replaced by heteroatoms, and the ring having optionally type R′ side chains, in which R′ is as defined above; or one of the following groups: 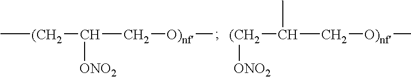
wherein nf′ is an integer of from 1 to 6, preferably of from 1 to 4 carbon atoms, 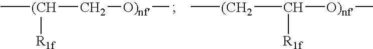
wherein R_{1f}=H, CH₃ and nf′ is as defined above,
Y_{Ar} and is selected from: 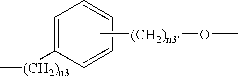
wherein n3 is an integer of from 0 to 3 and n3′ an integer of from 1 to 3; 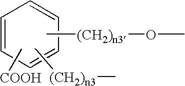
wherein n3 and n3′ are as defined above.

2. Use according to claim 1, wherein the B precursor is selected from:
amino acids, preferably selected from L-carnosine (formula CI), anserine (CII), selenocysteine (CIII), selenomethionine (CIV), penicillamine (CV), N-acetylpenicillamine (CVI), cysteine (CVII), N-acetylcysteine (CVIII), glutathione (CIX) or esters thereof, preferably ethyl or isopropyl ester, aspartic acid (PI), hystidine (PII), 5-hydroxytryptophan (PIII): 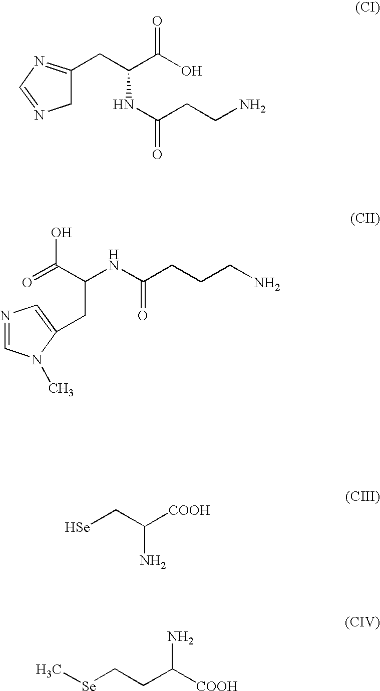 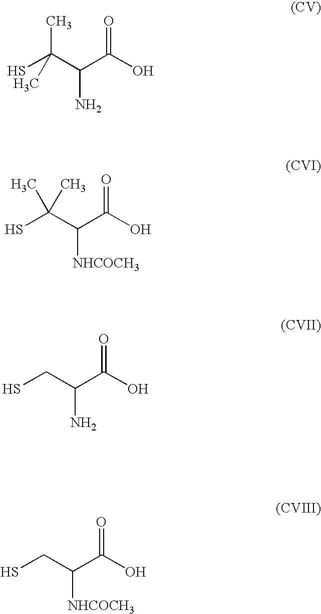 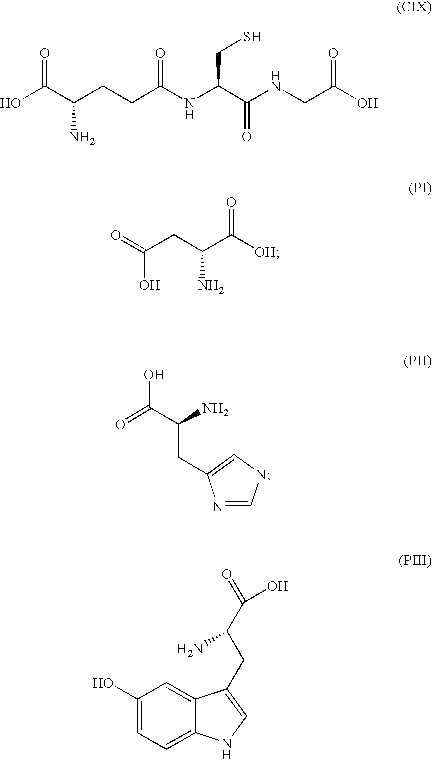
hydroxy acids, preferably selected from the following: gallic acid (DI), ferulic acid (DII), gentisic acid (DIII), citric acid (DIV), caffeic acid (DV), dihydroxycaffeic acid (DVI), p-coumaric acid (DVII), vanillic acid (DVIII), dihydroxymaleic acid (NIII): 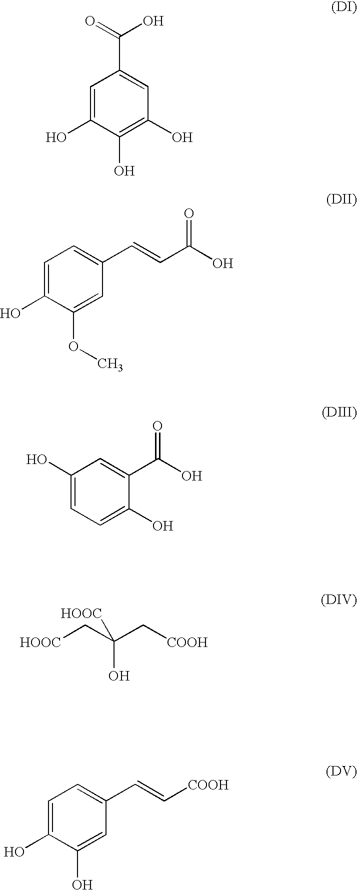 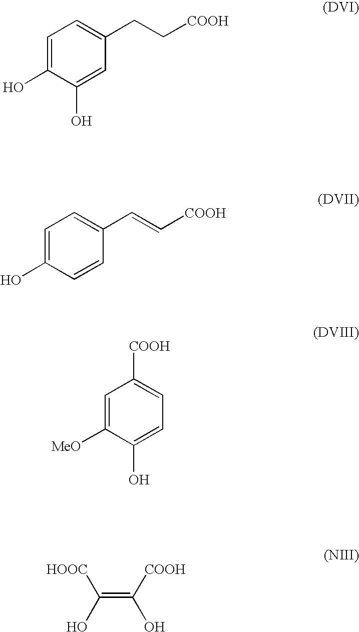
mono or polyalcohols preferably selected from the following: nordihydroguaiaretic acid (EI), quercetin (EII), catechin (EIII), kaempferol (EIV), sulfuretin (EV), hydroquinone (EVIII), gossypol (EIX), reductic acid (EX), methoxyhydroquinone (EXI), hydroxyhydroquinone (EXII), propyl gallate (EXIII), 3,5-di-ter.butyl-4-hydroxybenzyl-thioglycolate (EXXIV), saccharose (EC), ascorbic (ECI) and isoascorbic (ECII) acid, p-coumaric alcohol (ECIII), 4-hydroxy-phenylethyl alcohol (ECIV), conyferil alcohol (ECV), 2-thiouracil (QI), 2-mercaptoethanol (QII)

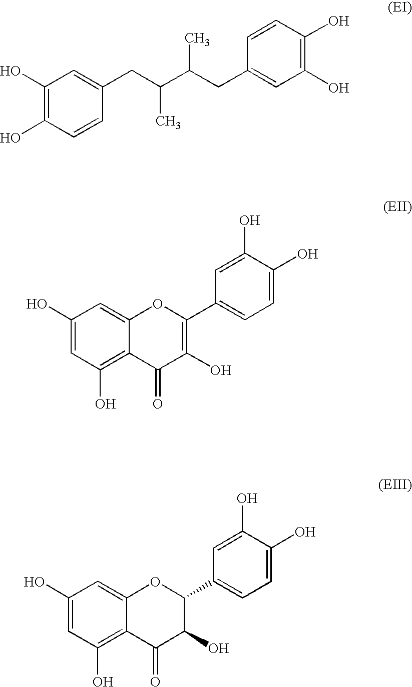

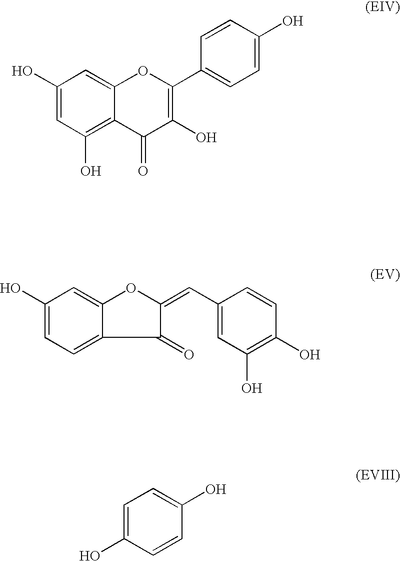

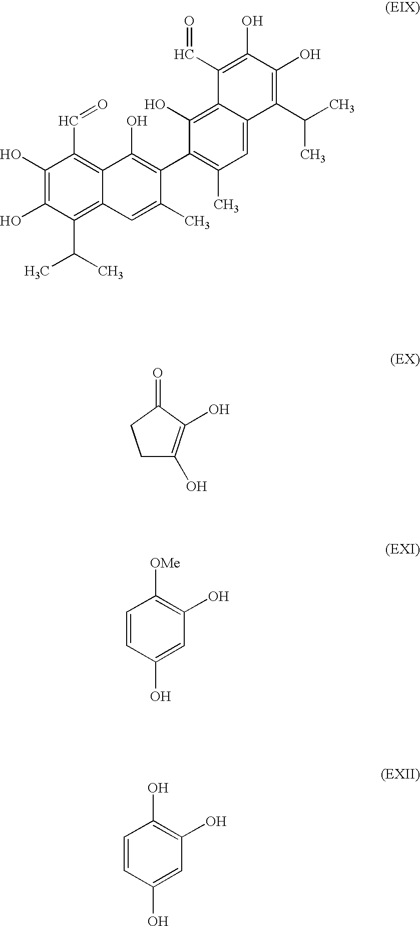

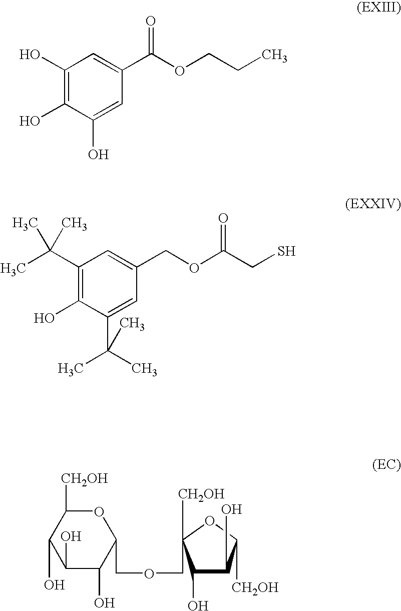

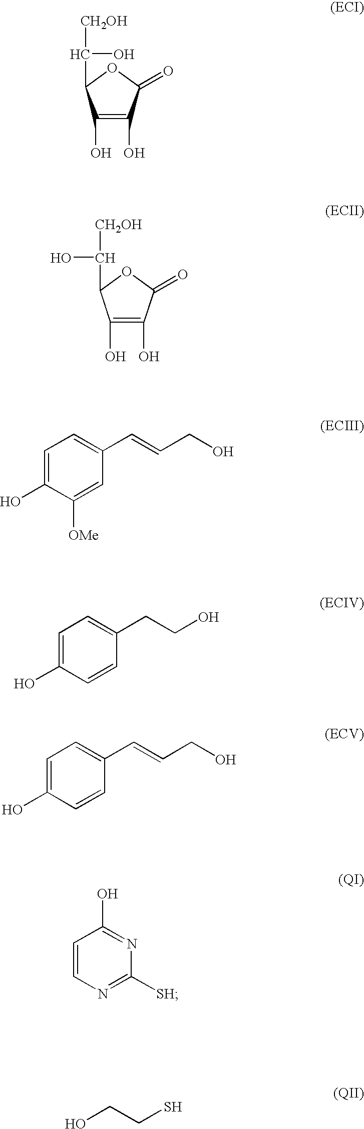

3. Use according to claim 1, wherein Y³ of radical C is selected from the following bivalent radicals:

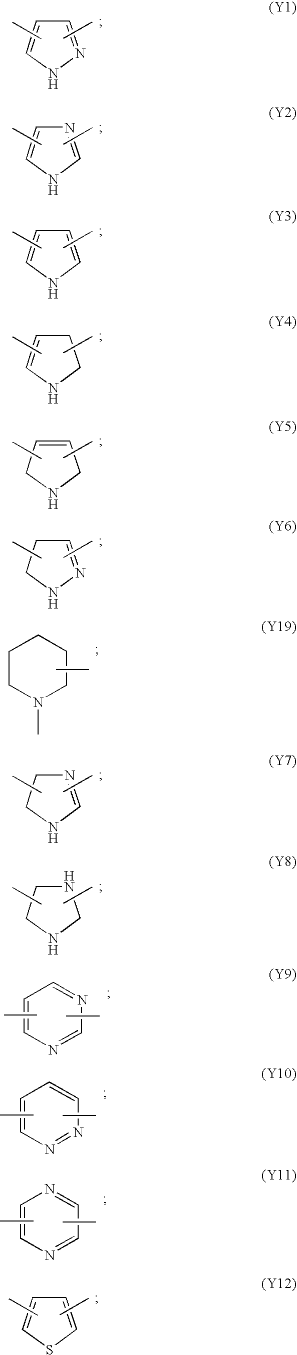

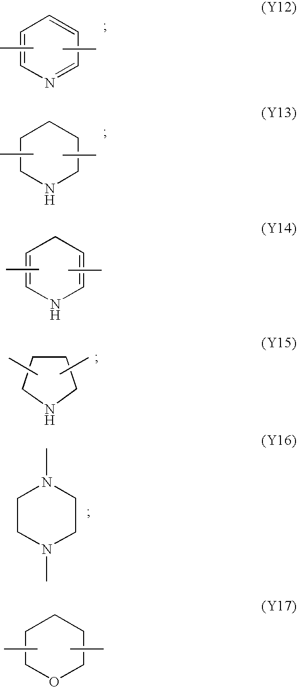

4. Use according to claim 3, wherein Y³ has the following meanings: (Y12), with both the free valences in ortho position as to the nitrogen atom; (Y16) with both the free valences attached to the nitrogen atoms; (Y1), 3,5-disostituted pyrazole; (Y19), wherein the free valence is para-standing on the ring as to the nitrogen atom.

5. Use according to claim 1, wherein b₀=c₀=1 in formula (I), B results from precursor ferulic acid, Y=Y₀ selected from alkylenoxy —R′O—, R′ containing preferably from 3 to 6 carbon atoms.

6. Use according to claim 5, wherein the compound of formula (I) is (3α,5β,7β)-3,7-dihydroxycholan-24-oic acid 2-methoxy-4[3-[4-(nitrooxy)butoxy]-3-oxo-1-propenyl]-phenyl ester having the following

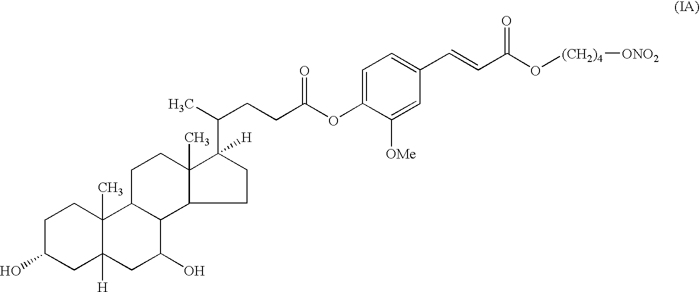

7. Use according to claim 1, wherein in formula (I) b₀=0, Y=Y₀ selected from alkylenoxy —R′O—, R′ containing preferably from 3 to 6 carbon atoms or Y=Y_{Ar}.

8. Use according to claim 7, wherein the compound of formula (I) is (3α,5β,7β)-3,7-dihydroxycholan-24-oic acid 4-(nitrooxy)-butyl ester of formula:

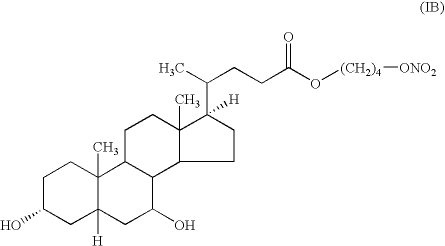

9. Compounds of formula (I) wherein b₀=0, Y=Y₀ selected from alkylenoxxy —R′O—, R′ containing preferably from 3 to 6 carbon atoms or Y=Y_{Ar}.

10. The compound of formula (I), that is (3α,5β,7β)-3,7-dihydroxycholan-24-oic acid 4-(nitrooxy)-butyl ester of formula: [chemical structure]
